(19) United States
(12) Patent Application Publication
(10) Pub. No.: US 2019/0217025 A1
(43) Pub. Date: Jul. 18, 2019

(54) Medical Instruments

(71) Applicant: Bruce H. Levin, Oceanside, NY (US)

(72) Inventor: Bruce H. Levin, Oceanside, NY (US)

(21) Appl. No.: 16/247,515

(22) Filed: Jan. 14, 2019

Related U.S. Application Data
(60) Provisional application No. 62/617,083, filed on Jan. 12, 2018.

(51) Int. Cl. A61M 11/00; A61M 15/00; A61M 15/08

(52) U.S. Cl. CPC A61M 11/003; A61M 11/007; A61M 11/008; A61M 25/0074; A61M 15/08; A61M 2206/20; A61M 15/009

(57) ABSTRACT

The present invention provides various medical instruments for delivery of nasal medication. It provides devices for influencing fluid flow around and/or inside a device. It also provides improved drug delivery systems to administer a desired dosage to the nasal epithelia, SPG, or a predetermined area.

### RELATED APPLICATIONS

This application claims priority to U.S. Provisional Patent Application Ser. No. 62/617,083 filed Jan. 12, 2018 titled the same and incorporation herein as if set out in full.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

The present invention relates to medical instruments for delivery of nasal medication. In other embodiments, the present invention provides devices for influencing fluid flow around and/or inside a device. There is also a need for unproved drug delivery systems to administer a desired dosage to the nasal epithelia or a predetermined area such as the sphenopalatine ganglion (hereinafter, the “SPG”) and the dorsal nasal nerve.

### BRIEF DESCRIPTION OF THE DRWINGS

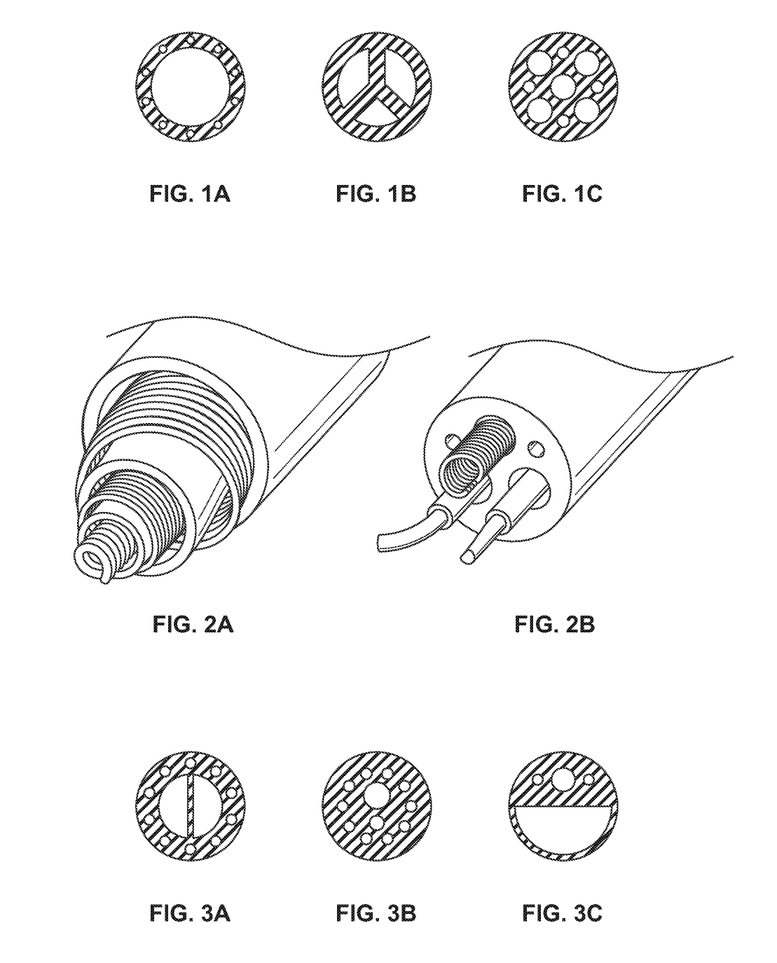

FIGS. 1A, 1B and 1C show three embodiments of the cross sections of a multipore or partially occlusive tip.

FIGS. 2A and 2B show two embodiments of a multipore or partially occlusive tip.

FIGS. 3A, 3B and 3C show three embodiments of the cross sections of a multipore or partially occlusive tip.

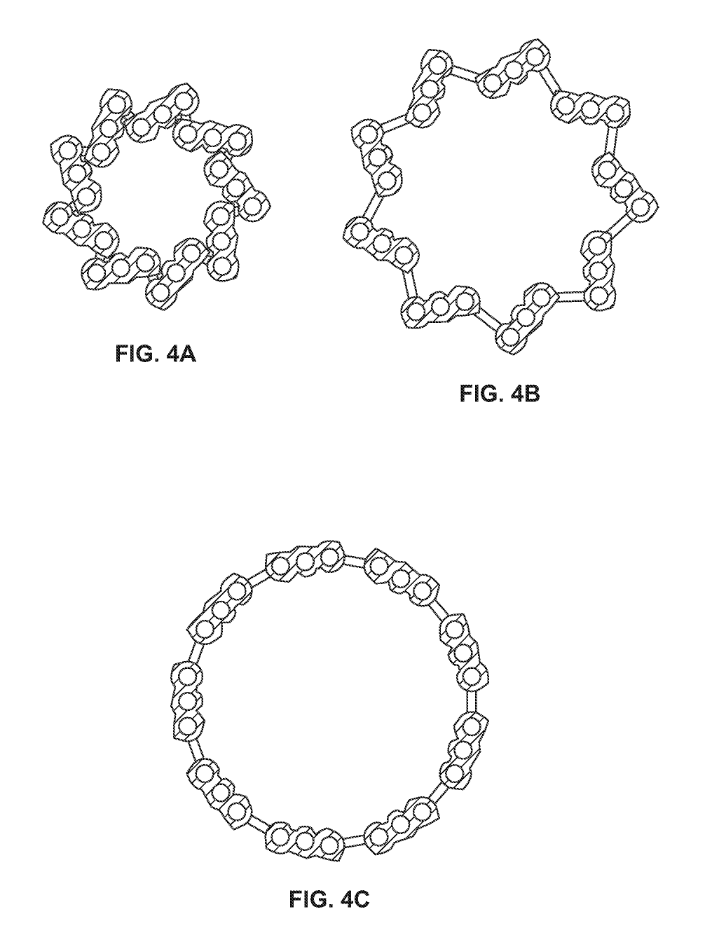

FIGS. 4A, 4B and 4C show a tip able to collapse and expand.

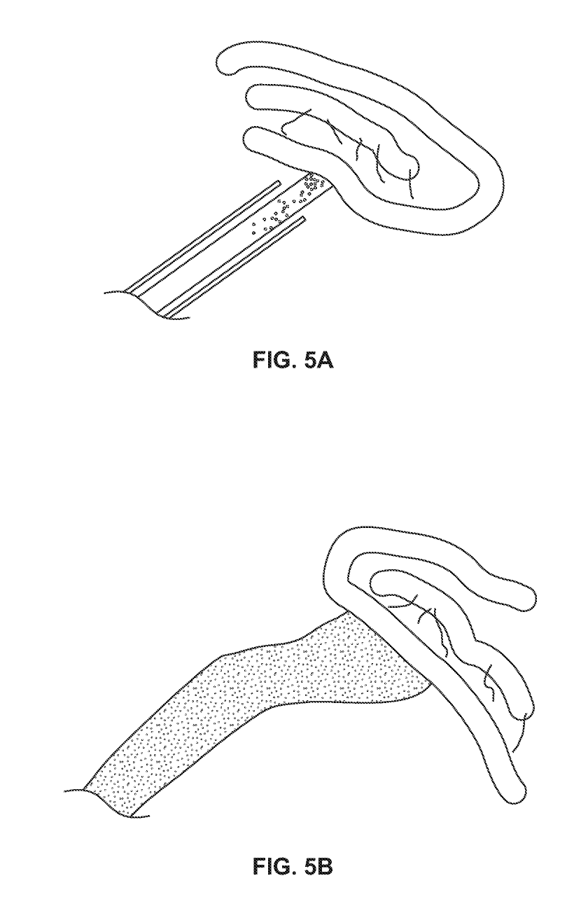

FIGS. 5A and 5B show two embodiments of medication applied at a desired location.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

In one embodiment, the present provides a medical device that is inserted into tissue that improves upon instruments configured to shoot a stream of fluid. In a preferred embodiment, the present invention applies higher volumes of fluid, such as a local anesthetic, to a targeted region such as the SPG.

In another embodiment, the present invention provides a multipore or partially occlusive tip, or other mechanism (such as shown in the drawings below), to effectively deliver in the form of a spray instead of a stream which will give better coverage with less wasted drip down.

FIG. 1 shows three embodiments of the cross sections of the tip. FIG. 1A represents an embodiment of the tip cross section, containing a large opening in the center and small openings surrounding the large centered opening. The centered large openings and the peripheral small openings and be open or close individually, to create a stream of mist or spray of nasal medication at a desired level. FIG. 1B represents another embodiment of the tip cross section, wherein the inside of the tip is divided to three openings and the three openings can be of equal or different sizes. Each opening can be open or closed as desired to control the amount and location of medication released by the device. FIG. 1C represents yet another embodiment of the tip cross section, wherein the tip contains openings vary in sizes to achieve the desired release and delivery of medication. Each opening can be open or closed to facilitate the desired result of release and delivery of medication.

FIG. 2 shows another two embodiments of the tip. FIG. 2A is a catheter tip that contains multiple layers of circular openings that can be extended out to achieve the desired result of release and delivery of medication. FIG. 2B shows a tip containing openings vary in sizes and shapes and each opening can be hollow or contains one or more parts. Each part can be of different shape and the parts can change direction, either as predetermined or change while using, to create a mist spray that covers contra laterally structures, bilateral structures of interest in the nasophayn.

In another embodiment, the tip can also release a spray or mist of medication in a fashion similar to a turbo to cover the desired locations and achieve the desired result of release and delivery of medication.

FIG. 3 shows three embodiments of the tip. FIG. 3A shows the tip with two half circle shaped openings in the center surrounded by a number of small openings. All of the openings can be open or close at the time of use to achieve the desired result of release and delivery of medication. The small openings help create a spray or mist of the medication, rather than traditional hollow tip that creates a stream when the medication is released from the tip. When the centered half-circle shaped openings are closed, the tip creates a small volume of spray or mist. When a larger volume of medication is needed, the centered half-circle shaped openings can be opened either on one side or both to achieve the desired result of release and delivery of medication. FIG. 3B is another embodiment of the tip that creates a more concentrated and focused mist or spray compare to the embodiment of FIG. 3A. Each of the openings within the tip can be open or close individually to achieve the desired result of release and delivery of medication. The porous openings can be larger than shown in FIG. 3B to create a larger volume of mist or spray. The control of the openings can create incremental application of the medication. FIG. 3C is yet another embodiment of the tip that has half of the tip maintain the traditional hallow structure and the other half have numerous porous openings that creates a spray or mist, stream, or a combination thereof, to achieve the desired result of release and delivery of medication.

FIG. 4 is yet another embodiment of the tip, wherein the tip can be folded shown in FIG. 4A when entering the nasal passage and later expand at desired location, as shown in FIG. 4B, FIG. 4C, or any state between the completely closed state shown in FIG. 4A to completely expanded state shown in FIG. 4C, to achieve the desired result of release and delivery of medication.

The porous openings in the tip can have be arranged to achieve the desired result of release and delivery of medication. The openings can be smaller in diameters toward the center of the tip and bigger in diameters towards the peripheral of the tip, or vice versa.

The geometry of the pores may be centrally located. In other designs, the pores may be partially occluding insert or inserts which would result in a spray. In other embodiments, the distal tip may include a geometric shape or cylinder stuck in a distal location of a hose work to be producing a spray depending on its geometry or even presence of channels. There are also many variations that are possible, and the spray can be any density or even a mist if desired.

The SPG is nonhomogeneous and has varied structural geometry. When administering medication, there may be local mucosal epithelial edema, increased thickness of the mucosa, or overlying mucous which would impede mucosal penetration of the applied or delivered medication or substance through the epithelium into the desired dorsonasal neural or other structures or tissues. A specific amount of time is required for applied medication to be absorbed by and penetrate through the mucosal surface.

Therefore, applying medication as a single or multiple dose spray or typical stream delivery would result in a significant amount of the applied medication not being absorbed into the desired dorsonasal or other neural or other structure. Further, there would be a significant amount of applied medication being repelled by or accumulated on the mucosal surface. This would result in a decreased therapeutic effect as the optimum dose of medication would not be delivered to its intended target. Further, the accumulated medication could then drip or be inhaled into other structures, for example, the esophagus, trachea or auditory meatus increasing unpleasantness to the patient and increasing side effect risk profile. Hence, one or a series of effectively timed mist or fine spray deliveries would allow the overlying mucosa to in effect be ‘painted’ with the applied medication being absorbed more completely, avoiding mucosal pooling and decreasing the wastage of immediate subsequent application. In this manner, the desired dose can be obtained and the administered medication dose would more closely resemble the actual delivered therapeutic dose. Hence, this delivery modality increases efficacy and decreases waste or distribution to the pharynx, oral cavity, or non-designated poorly therapeutic locations.

Moreover, because the SPG and related neural structures, and other dorsal nasal neurostructural, as well as other neural structures involved in migraine, cluster or other headaches, vary in geometry and location, and because blocking a part of the SPG or given neural structure may, and often does, give incomplete relief of a headache episode in terms of location of pain, severity or quality of pain and related symptoms, there is a need to have a device which can deliver the agent or medication to more than a single locus on the SPG. Further, similarly, other targeted areas, such as sinus ostia, the ethmoidal area, other areas that can be targeted for delivery to the CNS, brain, meninges, or CSF, may have delivery devices of similarly concepts or sort. a single stream delivery pattern will often saturate a small area and result in significant drippage of agent from the desired area, favoring swallowing, aspiration and pooling of the agent in a suboptimal area.

To address the above-mentioned drawbacks, in another embodiment the present invention provides a medical instrument configured to involve metered or non-metered delivery through a multi or single lumen, with increments easily delivered during rotation, twisting and/or advancement forward or retraction backward during delivery. Rate of injection can be fast or slow. The support for this are patients who had headache pain in periorbital, temporal, and other locations. Typical SPG block only relieved periorbital complaint, not lateral. Head was rotated dependently, and then the other location pain decreased.

In yet another embodiment of the present invention, the medical instrument has one or more spray pore loci at one or more locations at the tip, or side of the delivery device. These may spray, or preferably mist deposit, the agent for better mucosal coverage and saturation of the area where the desired neural structures are located. This will avoid drippage and unwanted pooling of the agent and avoid delivery of the agent to non-desired locations or structures, such as the esophagus or ear canal, later of which can result in severe vertigo which can last for days. The misting is best done by slow delivery, rather than rapid squirting, of the agent by device limited rate of injection, squeezing, twisting, advancing tube within a tube or other mechanism. The meter delivery maybe accomplished with a mechanical or other mechanism known to OSITA such as in auto injectors, diabetes Trulicity, flatulent, or other type of medication which are injected subcutaneously.

The spray mechanism can have one or delivery tips with different pore sizes, shapes, arrays and distribution on the delivery device as shown in the below drawings. One or more mechanisms can be applied to a single lumen or plurality lumen.

In yet other embodiments, the present invention may include a medical instrument having a central intraluminal tube, solid geometric structure, internal vortex generator, screw like insert, vane or vanes, or solid, rifling, twist or simple small diameter bar or rod like structure with or without deflection or other insert to allow one or more modalities of stream, spray or mist to one or more loci in the nasal mucosa or other area.

In yet other environments a rotating, clicking, advancing or twisting mechanism can be used to select the desired spray delivery tip or array from the different ones located at different locations on the device. Similarly, these manipulations can be used to select or choose one or more mechanisms, nozzle heads, delivery modes or delivery loci such as a rotatable spray head to choose the delivery type.

Importantly, the exit lumen or spray mechanisms can be directed or aimed to more than one target area, simultaneously in parallel, or sequentially based on where the patients head pain remains.

These, or similar modifications can also be used to make an irrigation device for the sinuses or nasophayn, or to deliver agents to the ethmoidal plate, sinuses, olfactory nerve and the like, and to other body areas intravascularly, intracavitary, interorgan, intracranially, in the GI, GU, GYN or other body parts and locations. Any agent including biologics, antibodies, viruses, vaccinations, antibiotics, gene or gene components, chemotherapeutic or antibiotic or any other agent can be so delivered.

For example, in a patient having frontal and temporal migraine headache symptoms and delivery to the SPG via catheter approach only resolved the frontal headache, it was necessary to have the patient fully rotate her head to the side of her remaining headache and another dose of medication was given and this decreased that temporal headache. Unfortunately, higher volumes were needed, and the patient had a very bad taste in her mouth and some pharyngeal numbness which was uncomfortable.

Using one of the mechanisms described herein to direct the agent to more than one location would circumvent this issue and result in less wastage and suboptimal delivery of agent. The support for this are patients who had headache pain in periorbital, temporal, and other locations. Typical SPG block only relieved periorbital complaint, not lateral. Head was rotated dependently, and then the other location pain decreased.

By using a multipore or partially occlusive tip, or other mechanism, an effective delivery in the form of a spray instead of a stream which will give better coverage with less wasted drip down.

In yet other embodiments, the medication may be in a solid, semisolid gel, or semisolid form as shown in the drawings below.

FIG. 5A shows an embodiment where a medication is in contact with mucosa surrounding SPG, such as a Q-tip, to release the medication. FIG. 5B shows an embodiment wherein the claimed device, where the tip release medication through a larger volume. The medication is dissolved over time to saturate the area.

In this embodiment, release of the medication may be made by contacting it directly to tissue which may result in the release of medication on contact, slowly over seconds to minutes

Body heat or moisture in nasopharyngeal or nasal mucosa facilitates the release of the medication. An inner spongiform or other reservoir, either presoaked/loaded or saturated by user with syringe, bulb, puncturable seal or other mechanism may be used.

As a screw mechanism, hydraulic, or direct slide advance mechanism advances the inner reservoir, the increased pressure squeezes out increments of medication (Blue dots). May also be short cross section, or may be nozzle, or there may be a nozzle spray tip.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A device for applying a medication wherein the device contains a tip with multipore or partially occlusive openings for delivery of medication.

2. The device of claim 1 where delivery is in a spray pattern or patterns.

3. The device of claim 1 where delivery is in a stream pattern or patterns.

4. The device of claim 1 where delivery is in a fine spray or mist pattern.

5. The device of claim 1 where the delivery patterns are mixed.

6. The device of claim 1, wherein the openings are evenly distributed.

7. The device of claim 1, wherein the openings having same diameters.

8. The device of claim 1, wherein the openings are circular in shape.

9. The device of claim 1, wherein each individual opening can open and close.

10. The device of claim 9, wherein the openings are prefixed to open or close.

11. The device of claim 1, wherein the openings containing cylindrical structures.

12. The device of claim 12, wherein one or more of the cylindrical structures is bent at a predetermined angle.

13. The device of claim 12, wherein the cylindrical structures can open or close.

14. The device of claim 12, wherein one or more of the cylindrical structures are prefixed to open or close.

15. The device of claim 1, wherein the tip can expand and collapse.

16. The device of claim 1, wherein the device may provide for unit or multi dose delivery with fixed or disposable delivery devices.

17. The device of claim 1, wherein the e devices may be metered dosed.

18. The device of claim 1, further comprising one or more of a syringe or syringe like, compressible bulb, dropper, twist delivery, pressurized delivery, or fixed or adjustable autoinjector mechanism known to one skilled in the art to trigger or effect medication delivery for single or multiple aliquots in a given treatment session.

19. The device of claim 1, wherein the device may be of the type of one more devices used to delivery respiratory care, humidity or antiasthmatic medications to pulmonary patients with nasal prong, nasal mask or other delivery unit known to one skilled in the art

20. The device of claim 15, wherein the device is designed to fit comfortably with a partially occlusive unilateral or bilateral set intranares tube or cylinder, or a nasal mask geometrically or anatomically constructed to comfortably cover the lower portion or more of the nose or nares and allow ingress of desired medication or other substance.

21. The device of claim of 16 wherein the substance or medication to be delivered can be a local anesthetic, NSAID, Insulin, Antidiabetic medication, monoclonal antibody, vaccine, antibiotic, anti-sinus or anti pneumonia agent, mushroom derivative, cation or ion, mineral, organic compound, colloidal silver, herbal, homeopathic agent, humidifying agent, antiasthmatic agent, CBD, cannabis, or marijuana or similar agent, tobacco or derivative or any vaping agent.

22. The device of claim 1, wherein the delivered agent further comprising one or more of naloxone HCl, or other opioid antagonist or sedative antagonist.
